(19) United States
(12) Patent Application Publication
(10) Pub. No.: US 2006/0052603 A1
(43) Pub. Date: Mar. 9, 2006

(54) Process for preparing substituted phenoxy-acetic acids from phenols

(76) Inventors: Jiasheng Guo, Durham, NC (US); Richard T. Matsuoka, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/528,242

(22) PCT Filed: Sep. 17, 2003
(86) PCT No.: PCT/US03/29482

§ 371 Date: Mar. 17, 2005

Related U.S. Application Data
(60) Provisional application No. 60/412,147, filed on Sep. 19, 2002.

(51) Int. Cl. A61K 31/506; A61K 31/497; A61K 31/496; C07D 417/14; C07D 417/02

(52) U.S. Cl. 544/295; 544/357; 548/202; 544/369

(57) ABSTRACT

The present invention provides a process for preparing a compound of formula (I): 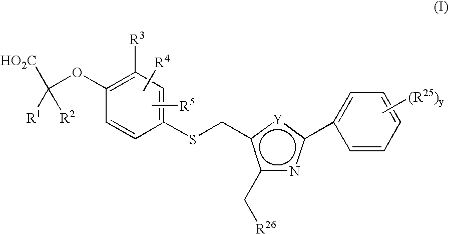
comprising the preparation of a compound of formula (II).

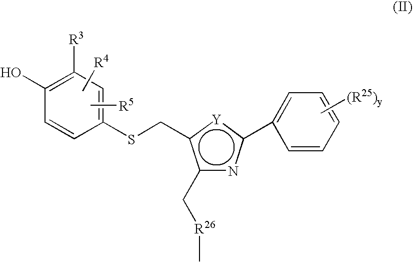

The present invention relates to novel method for preparing a certain compound. In particular, the present invention relates to preparing a compound that activates human peroxisome proliferator activated receptors (“hPPARs”).

Patent publication WO 02/059098 discloses compounds of formula (I) and pharmaceutically acceptable salts, solvates, and hydrolysable esters thereof wherein; 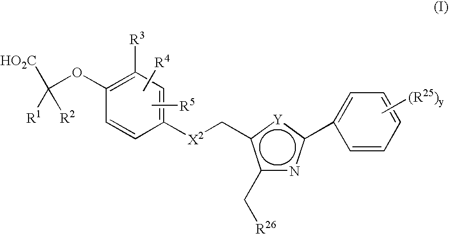
R¹ and R² are independently hydrogen or C₁₋₃ alkyl;
X² is O, S, or CH₂—;
R³, R⁴, and R⁵ are independently H, C₁₋₃alkyl, OCH₃, CF₃, OCF₃, allyl, CN, or halogen;
Y is S or O;
each R²⁵ is independently CH₃, OCH₃, OCF₃, CF₃, or halogen;
y is 0, 1, 2, 3, 4 or 5; and
R²⁶ is selected from the group consisting of the moieties A through K depicted below: 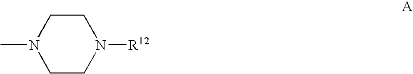
wherein R¹² is selected from the group consisting of C₁₋₆alkyl, C₁₋₆alkylenearyl, and the moieties depicted below in Group II, 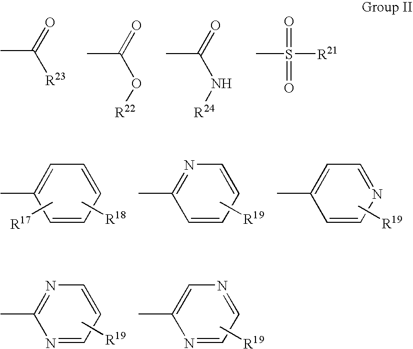
wherein R¹⁷ and R¹⁸ are independently hydrogen, halogen, hydroxy, —CN, C₁₋₆alkyl, C₁₋₆perfluoroalkyl, C₁₋₆acyl, —OC₁₋₆alkyl, perfluoroOC₁₋₆alkyl, or C₁₋₆hydroxyalkyl;
R¹⁹ is hydrogen or C₁₋₆alkyl;
R²¹ is C₁₋₆alkyl, —C₁₋₆alkylenearyl, aryl, or -aryl-heteroaryl;.
R²² is C₁₋₆alkyl, aryl, or —C₁₋₆alkylenearyl;
R²³ is C₁₋₆alkyl, C₃₋₆cycloalkyl, or aryl;
R²⁴ is C₁₋₆alkyl, —C₁₋₆alkylenearyl, C₃₋₆cycloalkyl, or aryl; 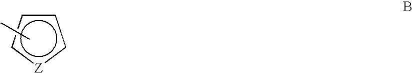
wherein Z is O, N or S (note that when Z is N, the depicted bond can be attached to he nitrogen in the ring as well as any of the carbons in the ring); 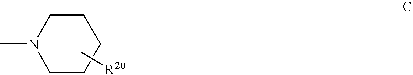
wherein R²⁰ is C₁₋₆alkyl, aryl, —OC₁₋₆alkyl, hydroxy, C₁₋₆hydroxyalkyl, or 1-alkoxyC₁₋₆alkyl; 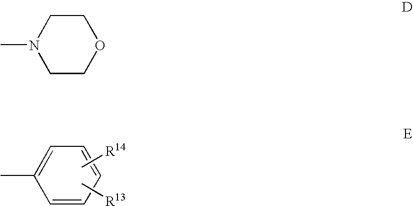
wherein R¹³ and R¹⁴ are independently hydrogen, halogen, CN, perfluroC₁₋₆alkyl, perfluroOC₁₋₆alkyl, C₁₋₆alkyl, —OC₁₋₆alkyl, —C₁₋₆alkyleneOC₁₋₆alkyl, —SC₁₋₆alkyl, or aryl; 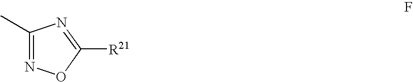
wherein R²¹ is independently as defined above; 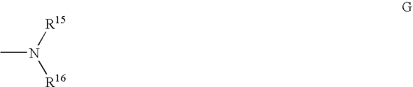
wherein R¹⁵ and R¹⁶ are independently hydrogen, C₁₋₆alkyl, C₃₋₆cycloalkyl optionally substituted with 1 or 2 C₁₋₃alkyl groups, or R¹² as defined above; [symbol]
   —(CH₂)nPh I
wherein n is 1-3
   —O—R²¹ J
wherein R²¹ is independently as defined above; and
   —S—R²¹ K
wherein R²¹ is independently as defined above. As used herein “aryl” or in any phrase or term including “aryl” such as “—C₁₋₆alkylenearyl”, the “aryl” means a phenyl group or a 5- or 6-membered heteroaryl group. As used herein “heteroaryl” means a 5- or 6-membered heteroaryl group. As used herein any such “aryl” or “heteroaryl” group may optionally be substituted with one or two substituents selected from the group consisting of halogen, CN, dimethylamino, perfluroC₁₋₆alkyl, perfluroOC₁₋₆alkyl, C₁₋₆alkyl, —OC₁₋₆alkyl, —C₁₋₆alkyleneOC₁₋₆alkyl, and —SC₁₋₆alkyl.

Methods for using and preparing the compounds of formula (I) are also disclosed in patent publication WO 02/059098. The compounds are useful for the treatment and prevention of a variety of diseases or conditions, for example diabetes and cardiovascular diseases and conditions including atherosclerosis, arteriosclerosis, hypertriglyceridemia, and mixed dyslipidaemia.

One of the preferred compounds disclosed and prepared in patent publication WO 02/059098 is 2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoic acid.

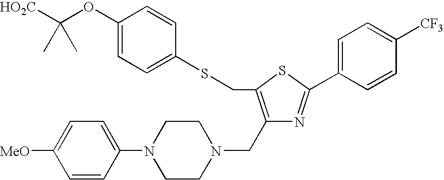

In the patent publication, two different routes of synthesizing this target molecule were disclosed. The initial synthesis of this molecule was a linear strategy consisting of fourteen chemical steps from 4-(trifluoromethyl)benzenecarbothioamide as summarized in Scheme IIa below.

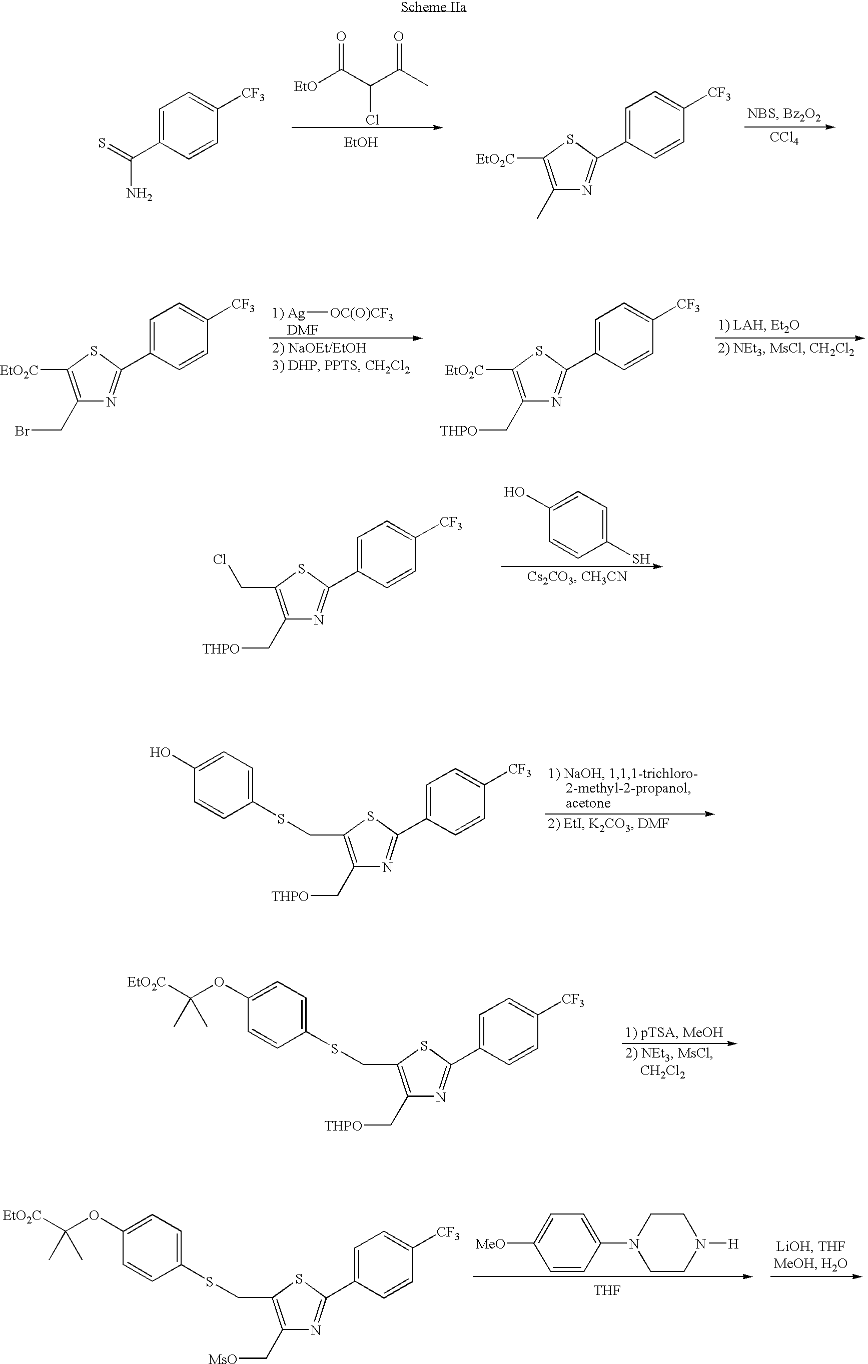

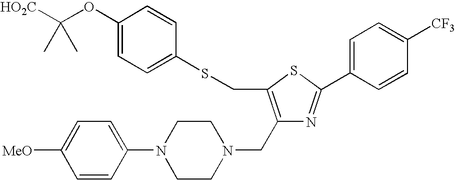

The second route of synthesizing the target molecule disclosed in patent publication WO 02/059098 was a convergent strategy. While this convergent synthesis consisted of a total of eleven chemical steps, the longest linear sequence involved only seven chemical steps as summarized in Scheme IIb below. The synthesis of ethyl 2-[4-(chlorosulfonyl)phenoxy]-2-methylpropanoate, which is used to make ethyl 2-methyl-2-(4-sulfanylphenoxy)propanoate by the zinc reduction reaction in the fourth step of this convergent synthesis, is shown in Scheme IIc.

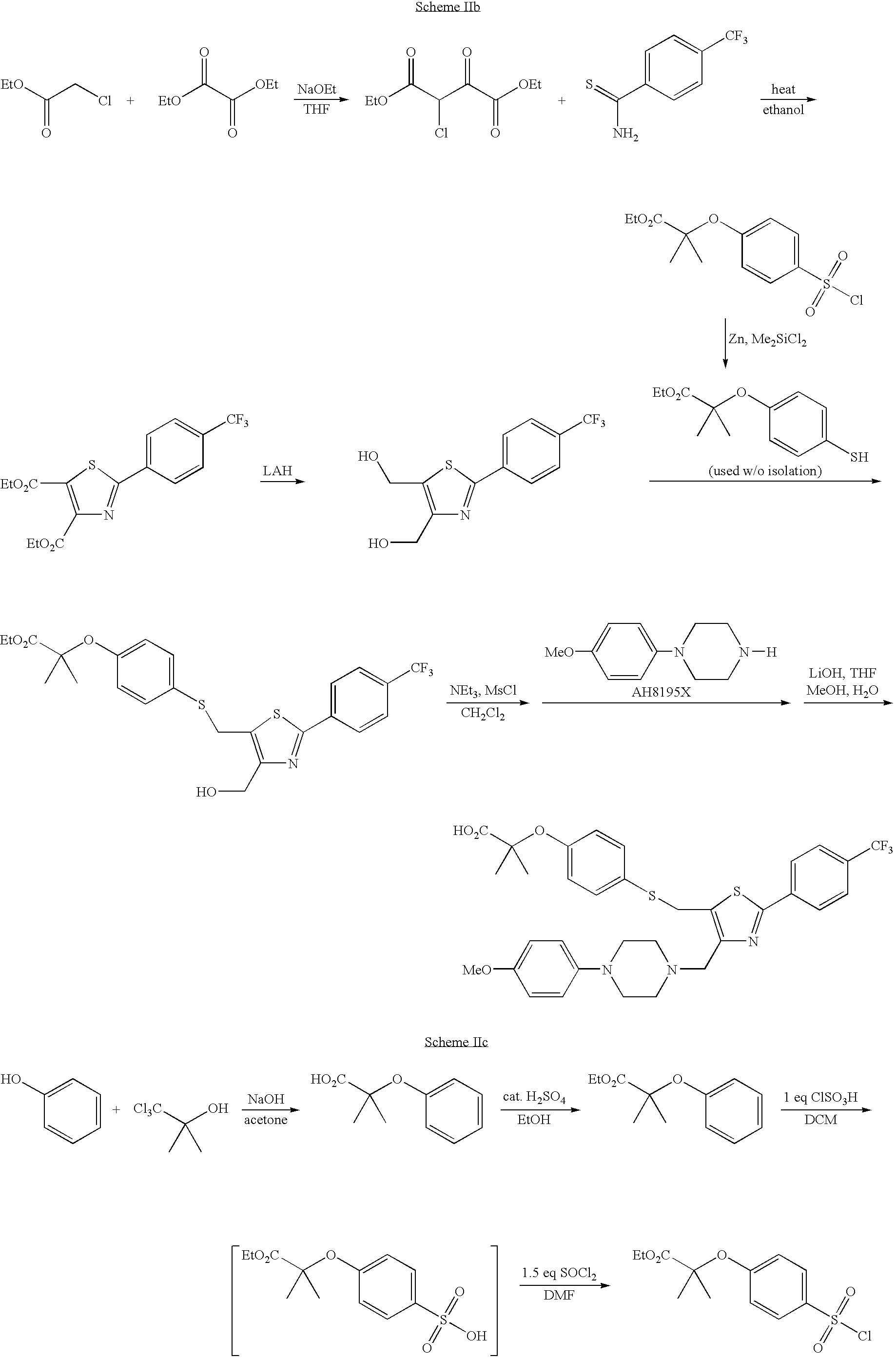

Briefly, in one aspect, the present invention provides a process for the preparation of a compound of formula (I) wherein X² is S, or a pharmaceutically acceptable salt, solvate, or hydrolyzable ester thereof, comprising the preparation of a compound of formula (II) wherein R³, R⁴, R⁵, R²⁵, R²⁶, Y, and y are as 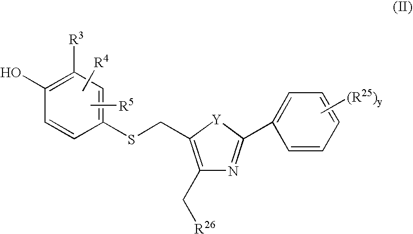
defined for formula (I). Preferred and most preferred compounds are as described in the above patent publication, with the proviso that X2 as defined in patent publication WO 02/059098 must be S for the purposes of this invention.

Briefly, in another aspect, the present invention provides a process for the preparation of a compound of formula (II) comprising the preparation of a compound of formula (III) wherein R³, R⁴, R⁵, R²⁵, Y, and y are as defined above.

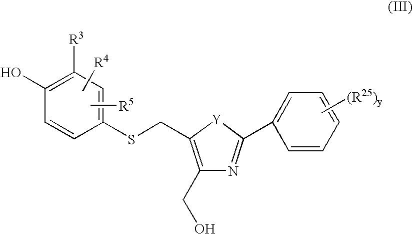

The compound of formula (III) may be prepared from the diol of formula (IV) as illustrated below.

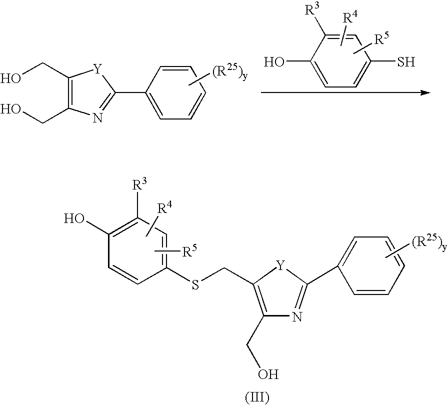

The preparation of compounds leading up to the processes of this invention and conversion of the compounds of formula (II) into compounds of formula (I) may be by known methods such as those described in patent publication WO 02/059098. For example, compounds of formula (II) may be converted to compounds of formula (I) by a process comprising treating a compound of formula (II) with a compound of formula (V). 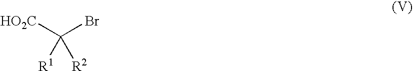
wherein R¹ and R² are as defined for formula (I).

The diol of formula (IV) may be prepared as described in patent publication WO 02/059098 or via the use of sodium borohydride or some other suitable reducing agent from the corresponding diester.

The improved processes of this invention are illustrated by the preparation of one of the preferred compounds disclosed and prepared in this patent publication.

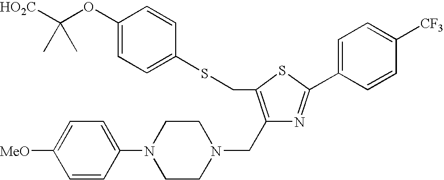

This compound can be prepared according to the processes of this invention as illustrated and summarized in Scheme I.

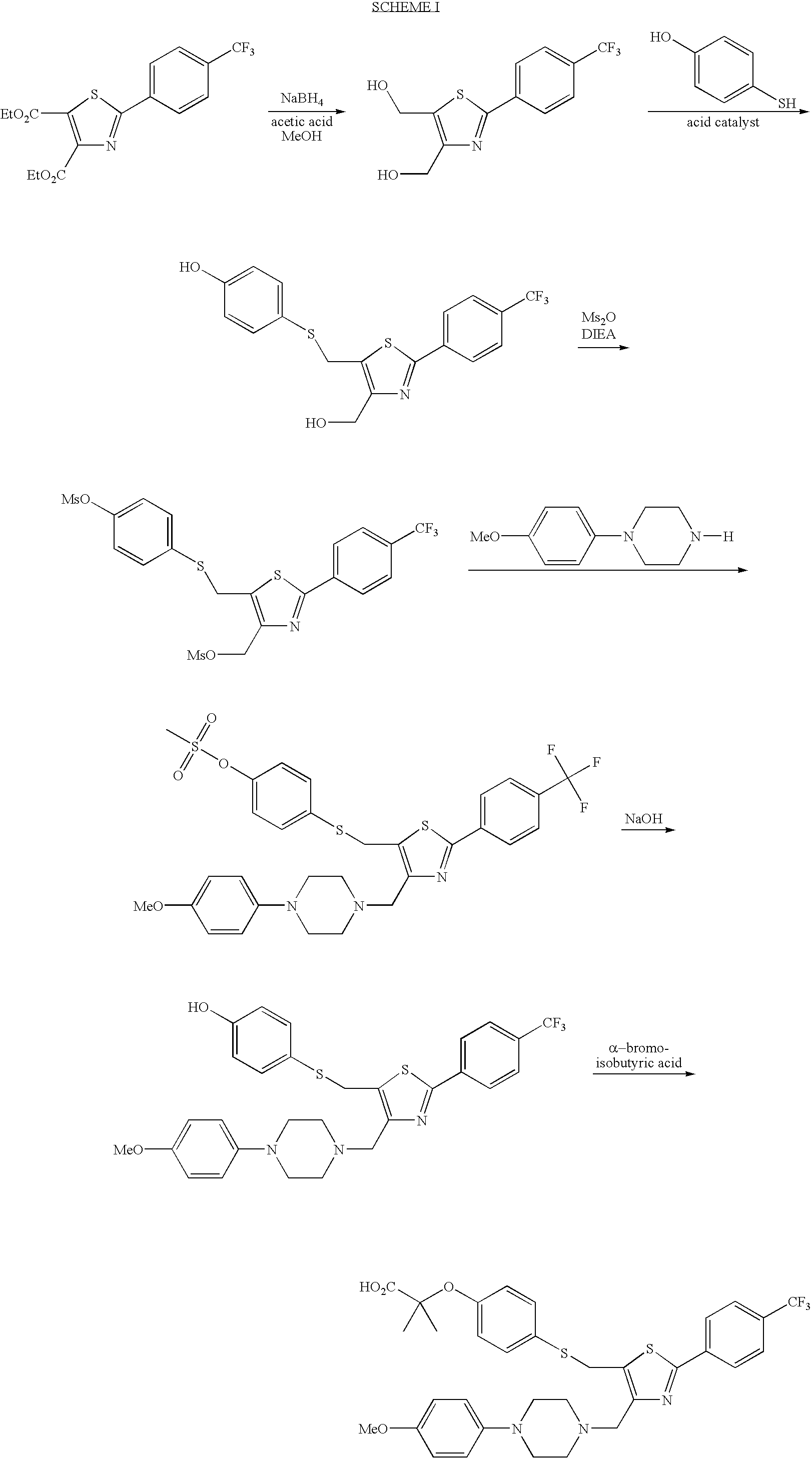

The reduction of diethyl 2-[4-(trifluoromethyl)phenyl]-1,3-thiazole-4,5-dicarboxylate to {5-(hydroxymethyl)-2-[4-(trifluoromethyl)phenyl)-1,3-thiazol-4-yl}methanol can be efficiently carried out as described in patent publication WO 02/059098, using lithium aluminum hydride as the reductant, or sodium borohydride as the reductant in the presence of methanol and acetic acid using tetrahydrofuran (THF) as the solvent.

The reaction between {5-hydroxymethyl-2-[4-(trifluoromethyl)phenyl]-1,3-thiazol-4-yl}methanol and 4-mercaptophenol can be successfully catalyzed by a variety of Lewis acids including zinc chloride in solvents such as isopropyl acetate, acetonitrile, or toluene, for example. The use of a Bronsted acid catalyst such as methane sulfonic acid instead of a Lewis acid, however, in solvents such as acetonitrile, tert-isobutyronitrile, or toluene afforded the desired product in consistently higher yield. Preferably, the reaction with 4-mercaptophenol is carried out in a one to one solvent mixture of acetonitrile and toluene using methane sulfonic acid as catalyst. The use of other Bronsted acids, such as hydrochloric acid in DME or triflouroacetic acid in acetonitrile, afforded the desired product in much lower yield.

The optimized conditions for carrying out the reaction between 4-[({4-(hydroxymethyl)-2-[4-(trifluoromethyl)phenyl]-1,3-thiazol-5-yl}methyl)sulfanyl]phenol and methansulfonic anhydride involved the use of N,N-diisopropylethylamine (“DIEA”) as base in dichloromethane. In addition to N,N-diisopropylethylamine and dichloromethane, other bases such as triethylamine and other solvents such as THF can also be used. The methanesulfonic anhydride reagent reacts at two centers of 4-[({4-(hydroxymethyl)-2-[4-(trifluoromethyl)phenyl]-1,3-thiazol-5-yl}methyl)sulfanyl]phenol, namely at the hydroxymethyl and the phenol functional groups. The reaction of the phenol functional group of 4-[({4-(hydroxymethyl)-2-[4-(trifluoromethyl)phenyl]-1,3-thiazol-5-yl)methyl)sulfanyl]phenol with methanesulfonic anhydride protects the phenol during the subsequent displacement of the aliphatic mesylate with 1-(4-methoxyphenyl)piperazine. If the phenol group is left unprotected during the reaction of the aliphatic mesylate with the piperazine, the phenol competes inter-molecularly with the piperazine for the aliphatic mesylate to produce unwanted dimeric-like side-products. After the reaction with the piperazine is complete, the mesylate-protecting group on the phenol is readily removed by treatment with a base, such as sodium hydroxide.

In the final step, the phenol group can be converted to the 2-methylpropanoic acid functionality, for example, by using the classic Bargellini reaction. The Bargellini reaction typically involves the use of 1,1,1-trichloro-2-methyl-2-propanol with bases such as sodium hydroxide, potassium hydroxide, or lithium hydroxide in solvents like acetone, THF, or ethanol. Preferably, this transformation is carried out with 2-bromoisobutyric acid using sodium hydroxide as base in methyl ethyl ketone (MEK) as described in the patent publication. Alternatively, the reaction with 2-bromoisobutyric acid can be performed with other bases including lithium hydroxide and in other solvents including acetone.

### EXAMPLES

The processes of this invention are illustrated by the following examples.

### Example 1

Example 1 is one embodiement of Scheme I described above.

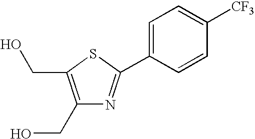

### {5-(Hydroxymethyl)-2-[4-(trifluoromethyl)phenyl]-1,3-thiazol-4-yl}methanol

Diethyl 2-[4-(trifluoromethyl)phenyl]-1,3-thiazole-4,5-dicarboxylate (200 g, 0.536 moles), methanol (142.5 g, 4.45 moles, 0.71 wt., 8.3 equiv.) and acetic acid (0.64 g, 0.01 moles, 0.0032 wt., 0.019 equiv.) were dissolved in tetrahydrofuran (530 mL, 2.65 volumes). This solution was added drop-wise to a stirred slurry of sodium borohydride (84.1 g, 2.22 moles, 0.42 wt., 4.1 equiv.) in tetrahydrofuran (1330 mL, 6.65 volumes) over about a 45 minute period. During the addition, there was gas evolution (H₂) and a 28° C. temperature rise. On completion of the addition, the reaction mixture was held at −45-50° C. for about 3 hours. Toluene (1330 mL, 6.65 volumes) was added to the cooled reaction mixture (20° C.), and the reaction mixture was quenched with aqueous 2N hydrochloric acid (1600 mL, 8 volumes). The layers were separated and the organic layer was concentrated (40° C., vacuum) to about one-half the original volume. The concentrated organic layer was then treated with hexanes (334 mL, 1.67 volumes) with stirring, and the product crystallized from solution, was filtered and washed with toluene/hexanes 1:1 (2×200 mL, 2×1 volume) and then dried in vacuo at 45° C. Yield: 120 grams, 77.4% of theory. ¹H NMR (300 MHz, CD₃OD): δ 8.12(2H; d, J=8.3 Hz), 7.85(2H, d, J=8.3 Hz), 6.54 (1H, br s), 5.72 (1H, br s), 4.77(2H, s), 4.79 (2H, s), 4.58 (2H, s), 4.77(2H, s).

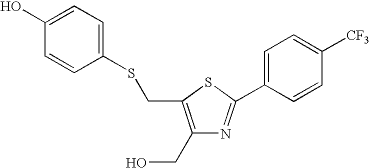

### 4-[({4-(Hydroxymethyl)-2-[4-(trifluoromethyl)phenyl]-1,3-thiazol-5-yl}methyl)sulfanyl]phenol

{5-Hydroxymethyl-2-[4-(trifluoromethyl)phenyl]-1,3-thiazol-4-yl}methanol (1380 g, 4.77 mol., 1 wt., 1.0 equiv.), 4-mercaptophenol (933 g, 7.39 mol., 0.67 wt., 1.55 eq.) in acetonitrile (6.9 L, 5 volumes) and toluene (6.9 L, 5 volumes) was slurried and methane sulfonic acid (747 g, 7.78 mol., 0.55 wt., 1.63 equiv.) was added. This solution was refluxed (85-86° C.) for about 13 hours. The reaction mixture was cooled to −25° C. and ethyl acetate (6.9 L, 5 volumes) was added. The reaction mixture was extracted with 10% potassium acetate (2×6.9 L, 2×5 volumes) (The pH of the aqueous layer after the second wash is about 7}. The layers were separated and the organic layer was filtered and concentrated (40° C., vacuum) to about one-half the original volume. Toluene (5.2 L, 3.8 volumes) was added and the mixture was reconstituted and re-concentrated a total of three times. The residual slurry was reconstituted with toluene (5.2 L, 3.8 volumes), and the crystallized product was filtered and washed with toluene (2×2.3 L, 2×2 volumes) and then dried in vacuo at −45° C. Yield: 1169 g, 61% of theory. ¹H NMR (300 MHz, DMSO-D₆): δ 9.69(1H, s), 8.07(2H, d, J=8.3 Hz), 7.84(2H, d, J=8.8 Hz), 7.23(2H, d, J=8.8 Hz), 6.72(2H, d, J=8.3 Hz), 5.21 (1H, t, J=5.7 Hz), 4.39 (2H, s), 4.33(2H, d, J=5.6)

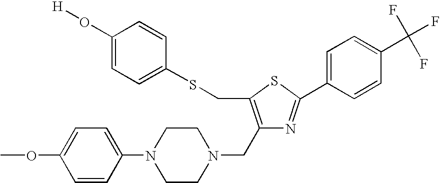

### 4-[({4-([4-(4-Methoxyphenyl)-1-piperazinyl]methyl]-2-[4-(trifluoromethyl)phenyl]-1,3-thiazol-5-yl}methyl)sulfanyl]phenol

A reaction vessel was charged with 4-[({4-(hydroxymethyl)-2-[4-(trifluoromethyl)phenyl]-1,3-thiazol-5-yl}methyl)sulfanyl]phenol (10 g, 1 wt., 1.0 equiv.), dichloromethane (50 mL, 5 volumes) and a 2 M solution of methansulfonic anhydride (2.4 equiv.) in dichloromethane (30 mL, 3 volumes) (an endotherm was observed). The slurry was cooled to −5-0° C. To the mixture was added N,N-diisopropylethylamine (10.5 mL, 1.05 volumes, 3.0 equiv.) at a rate such that the temperature was maintained below 0° C. (over 40 minutes). After the addition was complete (check completeness by HPLC analysis), to the mixture was added cold water (80 mL, 8 volumes) at a rate that the temperature was maintained below 10° C. The mixture was stirred for 5 min and then allowed to warm to room temperature. The organic layer was separated and concentrated to about 4 volumes. Tetrahydrofuran (THF) (70 mL, 7 volumes) was added and the mixture was concentrated to about 6 volumes and -then treated with 1-(4-methoxyphenyl)piperazine (9.7 g, 0.97 wt., 2.0 equiv.) (neat or in THF solution). The mixture was stirred at room temperature until complete judged by HPLC analysis (1 h). The solid piperazine salt was filtered and the solution was concentrated under vacuum .to approximately 3 volumes. Acetone (30 mL, 3 volumes), water (1 mL, 0.1 volume) and sodium hydroxide (3 g, 0.3 wt., 3 equiv., 20-40 mesh beads) were added successively. The mixture was stirred at room temperature. When the reaction was judged to be complete via HPLC analysis (1 h to overnight), the mixture was concentrated under vacuum to approximately 3 volumes. Ethyl acetate (80 mL, 8 volumes) was added followed by aqueous 1.0 N hydrochloric acid solution (50 mL, 5 volumes) (pH −6-7). The organic layer was separated and concentrated under vacuum to approximately 4 volumes. Toluene (40 mL, 4 volumes) was added and the mixture was concentrated again to approximately 4-volumes. Solids were allowed to precipitate. The solid was collected by filtration after cooling to room temperature, washed with toluene (10 mL, 1 volume), pulled to dryness under vacuum to afford an off-white solid. Yield: 12.1 g, 84% of theory, Purity: ˜99% AUC. ¹H-NMR (400 MHz, CD₃OD): δ 2.53 (4H, m), 2.98 (4H, m), 3.42 (2H, s), 3.70 (3H, s), 4.26 (2H, s), 6.71 (2H, d, J=8.6 Hz), 6.80 (2H, d, J=9.0 Hz), 6.90 (2H, d, J=9.0 Hz), 7.25 (2H, d, J=8.6 Hz), 7.73 (2H, d, J=8.2 Hz), 8.06 (2H, d, J=8.2 Hz).

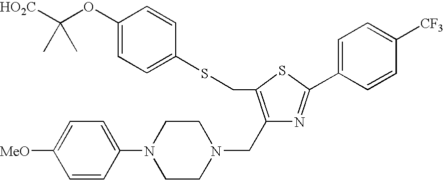

### 2-[4-[({4-{[4-(4-Methoxyphenyl)-1-piperazinyl]methyl}-2-[4-(trifluoromethyl)phenyl]-1,3-thiazol-5-yl}methyl)sulfanyl]phenoxy]-2-methylpropanoic acid

A flask was charged with 4-[({4-{[4-(4-methoxyphenyl)-1-piperazinyl]methyl}-2-[4-(trifluoromethyl)phenyl]-1,3-thiazol-5-yl}methyl)sulfanyl]phenol (20 g, 35.0 mmol, 1 wt., 1.0 equiv.), sodium hydroxide (6.3 g, 157.4 mmol, 0.32 wt., 4.5 equiv.), methyl ethyl ketone (MEK) (160 mL, 8 volumes), and water (2 mL, 0.1 volume). The mixture was heated with vigorous stirring to 50° C. and stirred for 3 h. A solution of 2-bromo-2-methylpropionic acid (11.7 g, 70.0 mmol, 0.59 wt., 2 equiv.) in MEK (40 mL, 2 volumes) was added drop-wise over a 1 h period to the reaction mixture at 50° C. After the addition was complete, stirring at 50° C. was continued for 2 h. Water (60 mL, 3 volumes) was then added to the reaction mixture and the resultant biphasic solution was cooled to room temperature. The biphasic reaction mixture .was stirred for 15 min. The stirring was stopped and the layers were allowed to separate. The layers were separated and the aqueous layer was discarded. The organic layer was treated with ethyl acetate (100 mL, 5 volumes) and aqueous 1 N HCl solution (35 mL, 1.6 volumes, 1 equiv.). [Note: The apparent pH of the mixture should be between 5 and 7; the pH may be adjusted if necessary.] The layers were allowed to separate and the aqueous layer was discarded. The organic layer was treated with aqueous 95% ethanol (60 mL) and seeded with 2-(4-[({4-{[4-(4-methoxyphenyl)-1-piperazinyl]methyl}-2-[4-(trifluoromethyl)phenyl]-1,3-thiazol-5-yl}methyl)sulfanyl]phenoxy)-2-methylpropanoic acid (0.5%, 100 mg, 0.005 wt., Form 1). The reaction mixture was stirred for 1 h and then the total volume of the mixture was reduced by 50% via distillation under reduced pressure (bath temp at 50° C.). During the course of the vacuum distillation, aqueous 95% ethanol (60 mL, 3 volumes) was added and removed twice. The resulting slurry was cooled to 10° C. and stirred at that temperature for 30-60 min. The slurry was filtered and the wet cake was washed with 95% ethanol (2×20 mL, 2×1 volume). The solid was dried under reduced pressure (20 in Hg) at 55-60° C. overnight. Yield: 18.9 g, 82% of theory; Purity: 99.6% AUC. ¹H NMR (400 MHz, CD₃OD): δ 8.08(d, 2H, J=8.24 Hz), 7.75(d, 2H, J=8.24 Hz), 7.25(d, 2H, J=8.61 Hz), 6.94(d, 2H, J=9.16 Hz), 6.82(m, 4H), 4.28(s, 2H), 3.72(s, 3H), 3.59(s, 2H), 3.16(t, 4H, J=4.94 Hz), 2.96(t, 4H, J=4.94 Hz), 1.54(s, 6H),

CHN Analysis: Theory (C, 60.26%; H, 5.21%; N, 6.39%) Found (C, 60.11%; H, 5.31%; N, 6.23%)

Unlike the processes described in patent publication WO 02/059098, every step of the synthesis of the target molecule, as described in this patent, is compatible with scale-up. In particular, the zinc reduction of ethyl 2-[4-(chlorosulfonyl)phenoxy]-2-methylpropanoate to and ethyl 2-methyl-2-(4-sulfanylphenoxy)propanoate can be prone, on large scale, to exhibit unpredictable and uncontrollable exotherms. In addition, the large-scale purification of ethyl 2-[4-(chlorosulfonyl)phenoxy]-2-methylpropanoate, the starting material in this zinc reaction, can be difficult since this material is an oil.

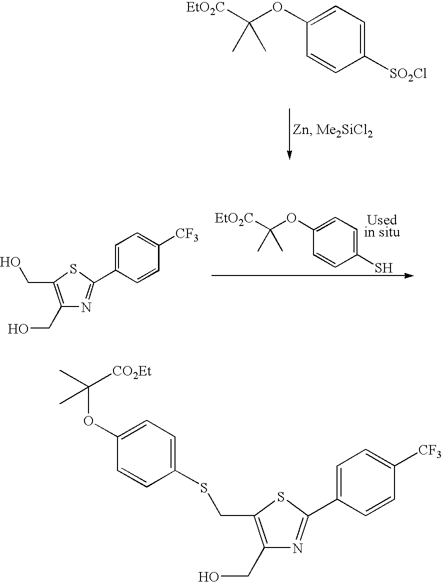

What is claimed is:

1. A process for the preparation of a compound of formula (I) or a pharmaceutically acceptable 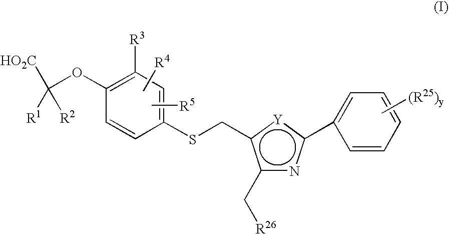
salt, solvate, or hydrolyzable ester thereof, comprising the preparation of a compound of formula (II) 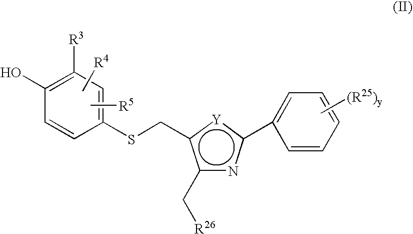
wherein:
R¹ and R² are independently hydrogen or C₁₋₃ alkyl;
R³, R⁴, and R⁵ are independently H, C₁₋₃alkyl, OCH₃, CF₃, OCF₃, CN, allyl, or halogen;
Y is S or O;
each R²⁵ is independently CH₃, OCH₃, CF₃, or halogen;
y is 0, 1, 2, 3, 4 or 5; and
R²⁶ is selected from the group consisting of the moieties A through K depicted below: 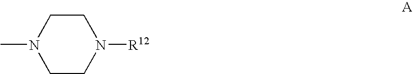
wherein R¹² is selected from the group consisting of C₁₋₆alkyl, C₁₋₆alkylenearyl, and the moieties depicted below in Group II, 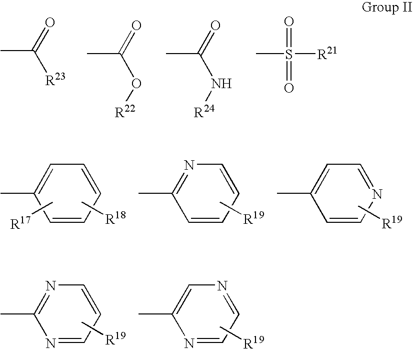
wherein R¹⁷ and R¹⁸ are independently hydrogen, halogen, hydroxy, —CN, C₁₋₆alkyl, C₁₋₆perfluoroalkyl, C₁₋₆acyl, —OC₁₋₆alkyl, perfluoroOC₁₋₆alkyl, or C₁₋₆hydroxyalkyl;
R¹⁹ is hydrogen or C₁₋₆alkyl;
R²¹ is C₁₋₆alkyl, —C₁₋₆alkylenearyl, aryl, or -aryl-heteroaryl;
R²² is C₁₋₆alkyl, aryl, or —C₁₋₆alkylenearyl;
R²³ is C₁₋₆alkyl, C₃₋₆cycloalkyl, or aryl;
R²⁴ is C₁₋₆alkyl, —C₁₋₆alkylenearyl, C₃₋₆cycloalkyl, or aryl; 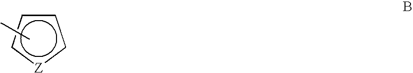
wherein Z is O, N or S (note that when Z is N, the depicted bond can be attached to the nitrogen in the ring as well as any of the carbons in the ring); 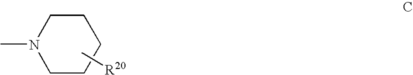
wherein R²⁰ is C₁₋₆alkyl, aryl, —OC₁₋₆alkyl, hydroxy, C₁₋₆hydroxyalkyl, or 1-alkoxyC₁₋₆alkyl; 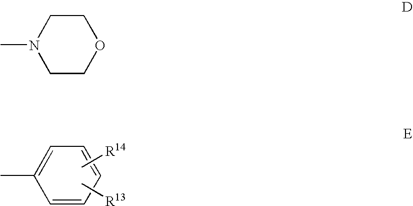
wherein R¹³ and R¹⁴ are independently hydrogen, halogen, CN, perfluroC₁₋₆alkyl, perfluroOC₁₋₆alkyl, C₁₋₆alkyl, —OC₁₋₆alkyl, —C₁₋₆alkyleneOC₁₋₆alkyl, —SC₁₋₆alkyl, or aryl; 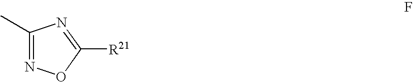
wherein R²¹ is independently as defined above; 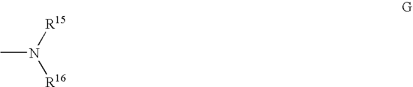
wherein R¹⁵ and R¹⁶ are independently hydrogen, C₁₋₆alkyl, C₃₋₆cycloalkyl optionally substituted with 1 or 2 C₁₋₃alkyl groups, or R¹² as defined above; [symbol]
—(CH₂)nPh I
wherein n is 1-3
—O—R²¹ J
wherein R²¹ is independently as defined above; and
—S—R²¹ K
wherein R²¹ is independently as defined above.

2. The method of claim 1 wherein R¹ and R² are independently H or CH₃, R³ is CH₃ or H, R⁴ and R⁵ are H, Y is S, y is 1 or 2, each R²⁵ is independently halogen or CF₃, R²⁶ is selected from the group consisting of 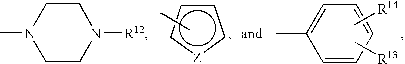
R¹³ and R¹⁴ are independently fluorine, bromine, phenyl, thienyl, CF₃, OCF₃, OCH₃, SCH₃, or t-butyl, R¹⁷ and R¹⁸ are independently hydrogen, OH, CN, OC₁₋₃alkyl, halogen, CF₃, COCH₃, CH(OH)CH₃, or OCF₃, R²¹ is phenyl optionally substituted by methyl or CN, —C₁₋₃-alkylenephenyl, or phenyl-5-methyl-1,2,4-oxadiazol-3-yl, R²² is C₁₋₃-alkyl, phenyl, or benzyl, R²³ is C₁₋₆alkyl, furanyl, thienyl, phenyl optionally substituted by a halogen a methoxy or a dimethylamino group, methoxymethylcyclopropyl, or C₃₋₆cyclalkyl, and R²⁴ is H, C₁₋₆alkyl, cyclohexyl, m-methoxyphenyl, p-fluorophenyl, or —CH₂CH₂phenyl.

3. The method of claim 1 wherein said compound of formula (I) is selected from the group consisting of:
2-methyl-2-{2-methyl-4-[({4-(3-thienylmethyl)-2-[4-(trifluoromethyl)phenyl]-1,3-thiazol-5-yl}methyl)sulfanyl]phenoxy}propanoic acid,
2-{4-[({4-{[4-(4-methoxyphenyl)-1-piperazinyl]methyl}-2-[4-(trifluoromethyl)phenyl]-1,3-thiazol-5-yl}methyl)sulfanyl]-2-methylphenoxy}propanoic acid,
{2-ethyl-4-[({4-{[4-(4-methoxyphenyl)-1-piperazinyl]methyl}-2-[4-(trifluoromethyl)phenyl]-1,3-thiazol-5-yl}methyl)sulfanyl]phenoxy}acetic acid,
2-{4-[({4-(4-methoxybenzyl)-2-[4-(trifluoromethyl)phenyl]-1,3-thiazol-5-yl}methyl)sulfanyl]-2-methylphenoxy}-2-methylpropanoic acid,
2-methyl-2-{4-[({4-{[4-(2-pyrazinyl)-1-piperazinyl]methyl}-2-[4-(trifluoromethyl)phenyl]-1,3-thiazol-5-yl}methyl)sulfanyl]phenoxy}propanoic acid,
2-{4-[({4-{[4-(4-methoxyphenyl)-1-piperazinyl]methyl}-2-[4-(trifluoromethyl)phenyl]-1,3-thiazol-5-yl}methyl)sulfanyl]-2-methylphenoxy}-2-methylpropanoic acid,
2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoic acid,
2-methyl-2-{2-methyl-4-[({4-[4-(trifluoromethoxy)benzyl]-2-[4-(trifluoromethyl)phenyl]-1,3-thiazol-5-yl}methyl)sulfanyl]phenoxy}propanoic acid,
2-{4-[((4-{([4-(4-isopropoxyphenyl)-1-piperazinyl]methyl)-2-[4-(trifluoromethyl)phenyl]-1,3-thiazol-5-yl}methyl)sulfanyl]-2-methylphenoxy}propanoic acid,
2-{2-methyl-4-[({4-{[4-(2-pyrimidinyl)-1-piperazinyl]methyl}-2-[4-(trifluoromethyl)phenyl]-1,3-thiazol-5-yl}methyl)sulfanyl]phenoxy}propanoic acid, and
pharmaceutically acceptable salts, solvates, and hydrolyzable esters thereof.

4. A method for the preparation of a compound of formula (II), comprising the preparation 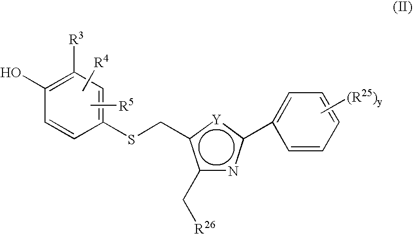
of a compound of formula (III) 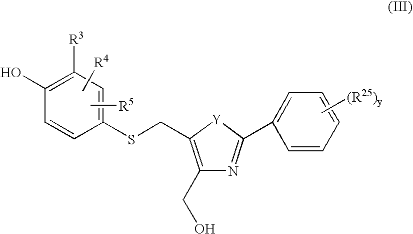
wherein:
R³, R⁴, and R⁵ are independently H, C₁₋₃alkyl, OCH₃, CF₃, OCF₃, CN, allyl, or halogen;
Y is S or O;
each R²⁵ is independently CH₃, OCH₃, CF₃, or halogen;
y is 0, 1, 2, 3, 4 or 5; and
R²⁶ is selected from the group consisting of the moieties A through K depicted below: 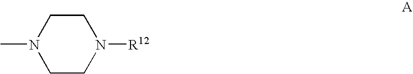
wherein R¹² is selected from the group consisting of C₁₋₆alkyl, C₁₋₆alkylenearyl, and the moieties depicted below in Group II, 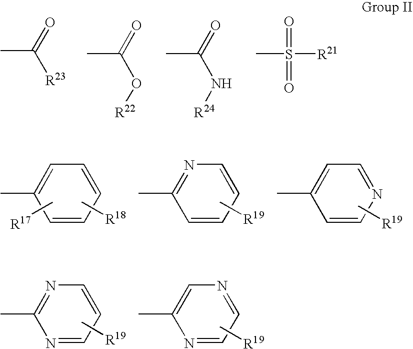
wherein R¹⁷ and R¹⁸ are independently hydrogen, halogen, hydroxy, —CN, C₁₋₆alkyl, C₁₋₆perfluoroalkyl, C₁₋₆acyl, —OC₁₋₆alkyl, perfluoroOC₁₋₆alkyl, or C₁₋₆hydroxyalkyl;
R¹⁹ is hydrogen or C₁₋₆alkyl;
R²¹ is C₁₋₆alkyl, —C₁₋₆alkylenearyl, aryl, or -aryl-heteroaryl;
R²² is C₁₋₆alkyl, aryl, or —C₁₋₆alkylenearyl;
R²³ is C₁₋₆alkyl, C₃₋₆cycloalkyl, or aryl;
R²⁴ is C₁₋₆alkyl, —C₁₋₆alkylenearyl, C₃₋₆cycloalkyl, or aryl; 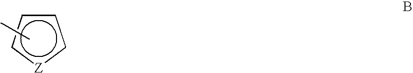
wherein Z is O, N or S (note that when Z is N, the depicted bond can be attached to the nitrogen in the ring as well as any of the carbons in the ring); 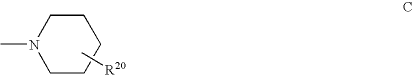
wherein R²⁰ is C₁₋₆alkyl, aryl, —OC₁₋₆alkyl, hydroxy, C₁₋₆hydroxyalkyl, or 1-alkoxyC₁₋₆alkyl; 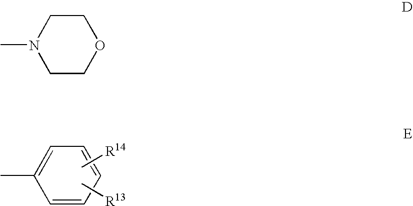
wherein R¹³ and R¹⁴ are independently hydrogen, halogen, CN, perfluroC₁₋₆alkyl, perfluroOC₁₋₆alkyl, C₁₋₆alkyl, —OC₁₋₆alkyl, —C₁₋₆alkyleneOC₁₋₆alkyl, —SC₁₋₆alkyl, or aryl; 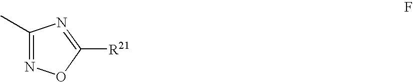
wherein R²¹ is independently as defined above; 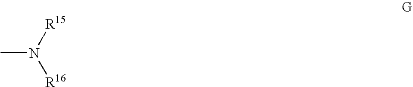
wherein R¹⁵ and R¹⁶ are independently hydrogen, C₁₋₆alkyl, C₃₋₆cycloalkyl optionally substituted with 1 or 2 C₁₋₃alkyl groups, or R¹² as defined above; [symbol]
—(CH₂)nPh I
wherein n is 1-3
—O—R²¹ J
wherein R²¹ is independently as defined above; and
—S—R²¹ K
wherein R²¹ is independently as defined above.

5. The method of claim 4 wherein R³ is CH₃ or H, R⁴ and R⁵ are H, Y is S, y is 1 or 2, each R²⁵ is independently halogen or CF₃, R²⁶ is selected from the group consisting of 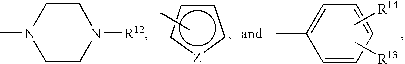
R¹³ and R¹⁴ are independently fluorine, bromine, phenyl, thienyl, CF₃, OCF₃, OCH₃, SCH₃, or t-butyl, R¹⁷ and R¹⁸ are independently hydrogen, OH, CN, OC₁₋₃alkyl, halogen, CF₃, COCH₃, CH(OH)CH₃, or OCF₃, R²¹ is phenyl optionally substituted by methyl or CN, —C₁₋₃alkylenephenyl, or phenyl-5-methyl-1,2,4-oxadiazol-3-yl, R²² is C₁₋₆alkyl, phenyl, or benzyl, R²³ is C₁₋₆alkyl, furanyl, thienyl, phenyl optionally substituted by a halogen a methoxy or a dimethylamino group, methoxymethylcyclopropyl, or C₃₋₆cyclalkyl, and R²⁴ is H, C₁₋₆alkyl, cyclohexyl, m-methoxyphenyl, p-fluorophenyl, or —CH₂CH₂phenyl.

6. The method of claim 1 further comprising the step of preparation of a compound of formula (III), wherein the compound of formula (III) is as defined in claim 4.

7. The method of claim 6 wherein wherein R¹ and R² are independently H or CH₃, R³ is CH₃ or H, R⁴ and R⁵ are H, Y is S, y is 1 or 2, each R²⁵ is independently halogen or CF₃, R²⁶ is selected from the group consisting of 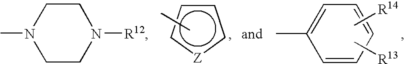
R¹³ and R¹⁴ are independently fluorine, bromine, phenyl, thienyl, CF₃, OCF₃, OCH₃, SCH₃, or t-butyl, R¹⁷ and R¹⁸ are independently hydrogen, OH, CN, OC₁₋₃alkyl, halogen, CF₃, COCH₃, CH(OH)CH₃, or OCF₃, R²¹ is phenyl optionally substituted by methyl or CN, —C₁₋₃alkylenephenyl, or phenyl-5-methyl-1,2,4-oxadiazol-3-yl, R²² is C₁₋₆alkyl, phenyl, or benzyl, R²³ is C₁₋₆alkyl, furanyl, thienyl, phenyl optionally substituted by a halogen a methoxy or a dimethylamino group, methoxymethylcyclopropyl, or C₃₋₆cyclalkyl, and R²⁴ is H, C₁₋₆alkyl, cyclohexyl, m-methoxyphenyl, p-fluorophenyl, or —CH₂CH₂phenyl.

8. The method of claim 7 wherein said compound of formula (I) is selected from the group consisting of:
2-methyl-{2-(2-methyl-4-[({4-(3-thienylmethyl)-2-[4-(trifluoromethyl)phenyl]-1,3-thiazol-5-yl}methyl)sulfanyl]phenoxy}propanoic acid,
2-{4-[({4-{[4-(4-methoxyphenyl)-1-piperazinyl]methyl}-2-[4-(trifluoromethyl)phenyl]-1,3-thiazol-5-yl}methyl)sulfanyl]-2-methylphenoxy}propanoic acid,
{2-ethyl-4-[({4-{[4-(4-methoxyphenyl)-1-piperazinyl]methyl}-2-[4-(trifluoromethyl)phenyl]-1,3-thiazol-5-yl}methyl)sulfanyl]phenoxy}acetic acid,
2-{4-[({4-(4-methoxybenzyl)-2-[4-(trifluoromethyl)phenyl]-1,3-thiazol-5-yl}methyl)sulfanyl]-2-methylphenoxy}-2-methylpropanoic acid,
2-methyl-2-{4-[({4-{[4-(2-pyrazinyl)-1-piperazinyl]methyl}-2-[4-(trifluoromethyl)phenyl]-1,3-thiazol-5-yl}methyl)sulfanyl]phenoxy}propanoic acid,
2-{4-[({4-{[4-(4-methoxyphenyl)-1-piperazinyl]methyl}-2-[4-(trifluoromethyl)phenyl]-1,3-thiazol-5-yl}methyl)sulfanyl]-2-methylphenoxy}-2-methylpropanoic acid,
2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoic acid,
2-methyl-2-{2-methyl-4-[({4-[4-(trifluoromethoxy)benzyl]-2-[4-(trifluoromethyl)phenyl]-1,3-thiazol-5-yl}methyl)sulfanyl]phenoxy}propanoic acid,
2-{4-[({4-{[4-(4-isopropoxyphenyl)-1-piperazinyl]methyl}-2-[4-(trifluoromethyl)phenyl]-1,3-thiazol-5-yl}methyl)sulfanyl]-2-methylphenoxy}propanoic acid,
2-{2-methyl-4-[({4-{[4-(2-pyrimidinyl)-1-piperazinyl]methyl}-2-[4-(trifluoromethyl)phenyl]-1,3-thiazol-5-yl}methyl)sulfanyl]phenoxy}propanoic acid, and
pharmaceutically acceptable salts, solvates, and hydrolyzable esters thereof.
